(19) United States
(12) Patent Application Publication
(10) Pub. No.: US 2013/0004552 A1
(43) Pub. Date: Jan. 3, 2013

(54) WATER DISPERSIBLE NONWOVEN FABRIC MATERIAL

(75) Inventor: Roberto Pedoja, Tampere (FI)

(73) Assignee: SUOMINEN CORPORATION, Tampere (FI)

(21) Appl. No.: 13/527,709

(22) Filed: Jun. 20, 2012

(30) Foreign Application Priority Data
Jul. 1, 2011 (EP) 11172330.0

(51) Int. Cl. D04H 13/00; B32B 38/04; A01N 25/08; B32B 37/02; A61K 9/70; A61K 8/02

(52) U.S. Cl. 424/401; 424/443; 424/409; 442/153; 156/280; 156/251; 15/104.93

(57) ABSTRACT

Non-woven multi-layered fabric materials which include two external layers made of continuous filaments of a water-soluble or water dispersible polymer having an internal layer of a water-absorbent, flushable material are provided. Methods for making such non-woven multi-layered fabric materials, are also provided.

### CROSS REFERENCE TO RELATED APPLICATION

This application claims priority to and benefit of European Patent Application No. 11172330.0 filed Jul. 1, 2011, the contents of which are incorporated by reference in their entirety.

### FIELD OF THE INVENTION

The present invention relates to the field of non-woven textile products and is applied to the manufacture of nonwoven fabric, particularly general nonwoven products, for various applications such as household use, personal and hygienic care. In particular, the present invention is applied to the manufacture of wipes and cleaning cloths.

### BACKGROUND OF THE INVENTION

Wet or impregnated wipes are used for a variety of purposes across several fields. For example, nonwoven fabric cloths are used for cleaning and may be impregnated with waxes or other cleaning solutions. Wet wipes are also used for personal care and may contain detergents, perfumes or even cosmetic lotions or creams.

These products are normally made from cellulose-based raw materials (100% cellulose or in any case a high cellulose content), such as viscose, cotton and the like, which are provided with absorbent properties.

High absorbent properties are necessary for the cloth to be wetted and retain a sufficiently high amount of the substance solution of the desired type (detergent, cosmetic and so on).

The above raw material fibres are generally hydroentangled or firmly bonded and are wetted with a detergent solution and/or perfume, so that they can be packaged ready for use.

Environment considerations however would discourage using these products. As the material used to make the wipes is not biodegradable, they are not flushable, so that they must be disposed as solid rubbish. This is not practical in most cases.

### SUMMARY OF THE INVENTION

An object of the present invention is therefore to provide a nonwoven fabric materials that overcome the above disadvantages.

A further object of the invention relates to processes for preparing such nonwoven fabric materials.

A further object of the invention is to provide, such as wipes and cleaning cloths, made of the nonwoven fabric material of the invention.

Further characteristics and the advantages of this invention will be better understood from the following detailed description of some embodiments thereof, which are provided by way of non-limiting examples.

### BRIEF DESCRIPTION OF THE FIGURES

FIG. 1 shows a schematic side view of a plant for manufacturing nonwoven fabric materials according to the present invention.

FIG. 2 shows a schematic side view of a plant for manufacturing nonwoven fabric materials according to another embodiment of the present invention.

FIG. 3 shows a schematic side view of a plant for manufacturing nonwoven fabric materials according to yet another embodiment of the present invention.

FIG. 4 shows a schematic side view of a plant for manufacturing nonwoven fabric materials according to still another embodiment of the present invention.

FIG. 5 shows a schematic side view of a plant for manufacturing nonwoven fabric materials according to a further embodiment of the present invention.

FIG. 6 shows a schematic side view of a plant for manufacturing nonwoven fabric materials according a further embodiment of the present invention.

FIG. 7 shows a graph reporting the results of a Tipping Tube test.

### DETAILED DESCRIPTION

Nonwoven fabric materials according to the present invention include multi-layered material comprising two external layers made of continuous filaments of a water-soluble or water-dispersible polymer and at least one internal layer of a water-absorbent, flushable material. In certain embodiments, such materials may be a tri-layered comprising one internal layer of water-absorbent material, one upper layer of continuous filaments of a water-soluble or water-dispersible polymer and one lower layer of continuous filaments of a water-soluble or water-dispersible polymer.

The nonwoven fabric material of the invention is a dry material. The term “dry material” as used herein means that the material of the invention is manufactured according to technologies and methods at a dry state, i.e. that do not involve the use of water. In particular, no bonding through hydroentanglement is performed. The water content of the inventive material can be attributed to the amount of water that is normally contained in or absorbed from the environment by the materials as such.

In certain embodiments, the dry material of the invention may have a water content of less than about 10% by weight and in other embodiments less than about 5% by weight. In one embodiment, the said water-absorbent material comprises cellulose pulp fibres.

In certain embodiments, the cellulose pulp fibres may have a length that may vary from substantially 0, i.e. cellulose powder, to 2.5 mm, for example, from 1 to 2 mm.

In one embodiment, the said water-soluble or water-dispersible polymer may be selected from polyvinyl alcohol (PVA), carboxymethyl cellulose, Guar Gum, alginate (also called alginic acid), starch, a co-polyester and mixtures thereof

Polyvinyl alcohol is a water-soluble, biodegradable synthetic polymer having a density of between 1.19 and 1.31 g/cc and a melting point of 230° C.

Carboxymethylcellulose is a cellulose derivative with carboxymethyl groups bound to some of the hydroxyl groups of the glucopyranose units. It is water-soluble and degradable.

Guar Gum is a galactomannan of natural origin and is biodegradable.

Alginate is a water-dispersible anionic polysaccharide having a density of 1.601 g/cc.

Water-dispersible copolyesters are synthetic products developed, for example, by Eastman Chemical. They can be used in nonwoven applications, such as those described in U.S. Pat. No. 6,087,550. These polymers are commercially available.

The continuous filaments may have a diameter between 0.5 dtex and 6.7 dtex, which in some embodiments may be between 0.9 and 2.5 dtex, and in other embodiments about 2 dtex.

In one embodiment, nonwoven fabric materials of the present invention may comprise additional dry substances such as, but not limited to: dry substances for household or for personal care, or dry substances for topic medical use and/or microcapsules for slow releasing of one or more of such substances.

Non-limiting examples of a substance for household application include: dry detergents and surfactants, waxes for ceramic or wooden floor, waxes for wood furniture surfaces, surface disinfectants, antibacterial, antiviral and/or antifungal products for household use, metal or wood polishing creams and cleaning formulations in general.

In certain embodiments, the dry detergent may be sodium alfa-olefin (C14-C16) sulphonate.

Non-limiting examples of substances for personal care include: formulations for personal hygiene and/or sanitization, skin creams or waxes, tanning creams, sunscreen formulations, insect repellent formulations, deodorants, perfumes, antibacterial, antiviral and/or anti-fungal formulations, make-up removing substances and cosmetic products in general. Non-limiting examples of dry substance for topic medical use include: skin disinfectants, skin antibacterial, antiviral and/or antifungal substances, cicatrizing formulations and in general any drug formulation that can be administered by topical application, or sanitizing compositions for medical facilities, appliances or devices.

The term “dry substance” as used herein refers to a substance having a water content less than about 10%, and in some embodiments less than about 5%.

Nonwoven fabric materials according to the present invention also may contain SAF (super-absorbent fibres) and/or SAP (super-absorbent polymers powder).

Super-absorbent polymers are commonly made from the polymerization of acrylic acid blended with sodium hydroxide in the presence of an initiator to form a poly-acrylic acid sodium salt. Other material also may be used, such as polyacrylamide copolymer, ethylene maleic anhydride copolymer, cross-linked carboxymethyl cellulose, polyvinyl alcohols copolymers, cross-linked polyethylene oxides and starch-grafted copolymer of polyacrilonitrile and many others. They are normally made by suspension polymerization or solution polymerization. SAP and SAF are commercially available.

Several types of additives also may be added to nonwoven fabric materials, such as: lubricating additives to give smoothness and easy processability; antistatic additives to prevent damaging electrostatic currents that may degrade the product, or at worst, reduce the productivity of the machine; hydrophilic additives; anti-foam additives to avoid the formation of foam which may be generated upon use.

Nonwoven fabric materials according to embodiments of the invention may have a weight/surface ratio (basis weight) ranging between about 30 and about 100 g/m², and in some embodiments between about 40 and about 60 g/m².

Nonwoven fabric materials of the invention may be manufactured according to a process that includes:

a) placing a first layer of spunbond filaments made of a water-soluble or water-dispersible polymer as defined above;

b) placing, on the layer of step a), a layer of water-absorbable fibres as defined above;

c) placing another layer of said spunbond filaments;

d) bonding the single layers and/or the multi-layered material at a dry state, to provide one or more bonding steps.

Such processes also may comprise one or more steps of consolidation or pre-consolidation of the single layers of continuous filaments and/or of the multi-layered material before the one or more bonding step.

The process may comprise a final step of winding the nonwoven fabric material to make a fabric bobbin.

The spunbond filaments can be produced in line with the formation of the inventive nonwoven fabric material or alternatively can be made on a separate production line.

The spunbond filaments may be produced through extrusion by spinnerets of the above defined polymer materials so as to form continuous filaments. These filaments, on output from the spinnerets, can be hit by a jet of compressed air that causes the elongation and the electrostatic charging thereof such as to cause a mutual repulsion causing them to fall randomly onto a conveyor belt. The continuous filament may be obtained by a spinning process by means of 1- to 5-orifices, preferably 2-3 orifices, spinner.

The pre-bonding consolidation of the single layers or of the multi-layered nonwoven fabric material can be accomplished by passing the layered material through two rollers of a compactor, embosser or calender.

It should be noted that as used herein term “compactor” or “embosser” refers to a device known per se, which has only the function of changing the surface of a nonwoven ply to obtain a slight consolidation (pre-consolidation) and in addition, in the case of the embosser, such as to form patterns, writings or drawings in relief In other words, the compactor would have a pre-consolidation function, actually weak, whereas the embosser would have a preconsolidation and ornamental function, thereby increasing the thickness of the ply. On the contrary, the conventional calender, though being provided with a similar general structure, has the basic function of consolidating and partially bonding the filaments composing the nonwoven material while minimizing or at most maintaining the ply thickness being laid down.

One of the two rollers, in a conventional calendar, is engraved, i.e. it has ribs in the form of dots or dashes evenly alternating with grooves. In particular, the ribs usually have a height between about 0.4 and about 0.6 mm, a free head with a contact surface for the filaments of about 0.88 mm² and a distribution so as to cover 19-23% of the surface of the roller. These combinations of features are responsible for a firm consolidation of the nonwoven ply.

In one embodiment, the nonwoven fabric materials may be in the form of a bonded web structure that comprises more than one web, for example, 3 to 9 webs. With a number of webs in the range specified above, a more isotropic textile structure, and accordingly a maximized spatial layout of the filaments can be achieved, which results in a maximized filament-water contact surface. Water droplets are thereby adsorbed by the structure within the small spaces resulting from the random distribution of the filaments.

The step of bonding of the single layers or of the multi-layered material may be accomplished by means of a so called dry-laid process. Dry-laying procedure may comprise providing a web comprising about 5 to about 30 w % of a bicomponent fibre and passing it into a dryer wherein the material is subject to temperatures of between about 120 and about 200° C. for a time generally between about 3 and about 15 seconds. The high temperature melts the low-temperature melting polymer of the bicomponent fibres, thus establishing bonding points throughout the web. This process, known as air through bonding, is advantageous if a softer and thicker web structure is desired, especially if a side-by-side bicomponent fibre is used.

In another embodiment, the single layers or the multi-layered material are bonded by a well-known chemical bonding process. In this case, the web structure is treated—for example by printing, powder or foam application—with a solid form of a latex polymer or a binder, in amounts ranging from about 5 to about 60 w %. The web structure so-treated is then cured, for example by heat treatment. Suitable binders may be selected from styrene-butadiene rubber, vinyl copolymers, vinyl acetate, styrenated or vinyl acrylates, polyvinylchloride. The binder formulation may comprise further ingredients, including: surfactants (to improve binder adhesion, stability and ability to be converted into a foam); external cross-linkers; defoamers (to minimize foam in the process); repellent agents; salts (to impart low flame response properties and to convey antistatic properties); thickeners (to control rheology of the binder liquid); catalysts (to promote curing and cross-linking); acids and bases (to control pH of the binder); dyes and pigments; fillers (to reduce binder tack and to lower cost); optical brighteners (to increase whiteness); sewing aids (to provide lubrication).

In another embodiment, bonding of the single layers or of the multi-layered material may be accomplished by means of a needle-punching process. The material may be mechanically entangled by means of a multiplicity of barbed metal needles which mechanically move in a rapid reciprocating fashion forwards and backwards through the web, in a direction essentially perpendicular to the plane of the web. This movement of the barbed needles causes filaments and fibres within the web to become entangled with nearby filaments and fibres, making the web a coherent, strong structure.

In certain embodiments, it may be convenient to slightly delay the water-solubility or water-dispersibility of the nonwoven fabric material to improve its handiness during use, when the material, in some applications, is wetted, without however impairing its flushability after use.

In such embodiments, the nonwoven fabric material obtained as described above may be treated with hydrophobic substances.

In certain embodiments, the material may be impregnated with a solution of a derivative of a fatty acid in a non-aqueous organic solvent. A solution of stearoyl chloride in toluene may be used as such solvent. Stearoyl chloride (CAS [112-76-5] is employed as a mixture of C18H35ClO and C16H31ClO.

This impregnation step may comprise, for example, soaking the nonwoven fabric material into the fatty acid derivative solution for 5 to 60 sec and drying it at about 100° C. for 5 to 30 minutes.

The nonwoven fabric material so-treated may contain from about 0.5 to about 10 w %, or in some embodiments from about 0.6 to about 5 w %, of hydrophobic substance.

An experiment was run to determine whether this treatment improves the resistance to water-solubility or water-dispersibility of the material (Tipping Tube test).

The wipe was introduced in a plastic tube filled with 700 mL of water. The tube was subjected to 240 circular rotations. The number of rotations was recorded for three points:

1st end point: the samples were broken in 1×1 cm pieces;

2nd end point: the samples were broken in 5×5 mm pieces;

3rd end point: the samples were fully dispersed.

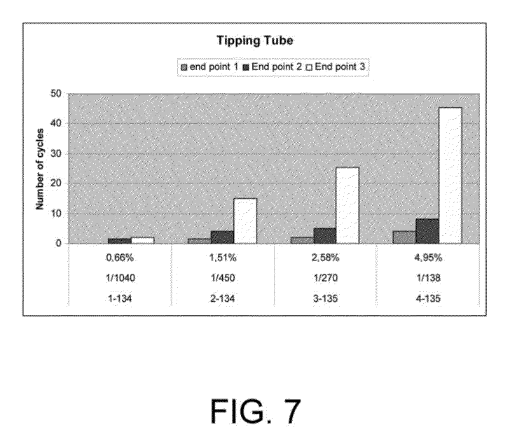

The results are reported in FIG. 7.

As shown, hydrophobic treatment increases significantly the resistance of the sample to the water-dispersion.

The process of the invention also may comprise one or more steps of adding to the single layers and/or to the nonwoven fabric material, before or after the bonding step, the additional substances described above.

The single layers or the non-woven fabric material may be impregnated with such substances by any known method. In particular, delivery of the substances on the web by means of suitable nozzles can advantageously be used. For creams and waxes, hot melt delivery can advantageously be used, due to the high viscosity of such substance forms.

In certain embodiments, processes according to the present invention may include one or more of the following final steps to increase or add additional characteristics to the end product: coloring or finishing of a chemical nature as anti-pilling treatments and hydrophilic treatments, antistatic treatments, improvement of flame proof properties, substantially mechanical treatments such as napping, sanforizing, and emerizing.

The invention will be described further by means of exemplary embodiments, with reference to the attached drawings. The drawings do not show the stations for adding the above defined additional substances. Such stations, however, are conventional and can be positioned at any point along the production line, preferably before the last bonding step.

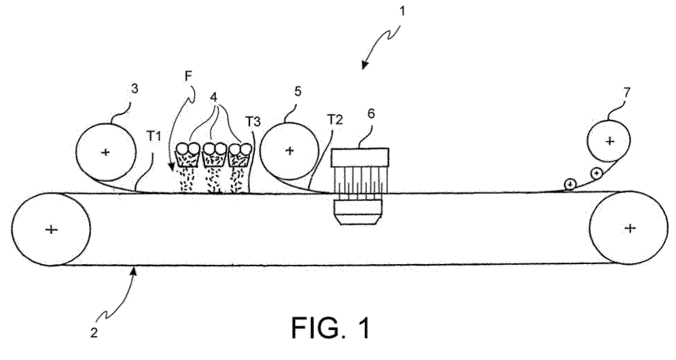

FIG. 1 shows a first embodiment of a production line 1 for manufacturing material according to the present invention.

On a transporting means 2, such as a conveyor belt, a layer T1 of consolidated spun-bond continuous filaments may be unwound from a feeding bobbin 3.

Subsequently, on the said layer T1 water-absorbent fibres F, such as cellulose pulp fibres, may be laid through suitable dispensing means 4 to form a layer T3.

After the deposition of said water-absorbent fibres, a second layer T2 of consolidated spunbond continuous filaments may be unwound and laid on the previous layers, after which the tri-layer material may be passed through dry bonding means 6 (shown in FIG. 1 as needle-punching means).

Finally, the bonded nonwoven fabric material may be wound on a receiving bobbin 7.

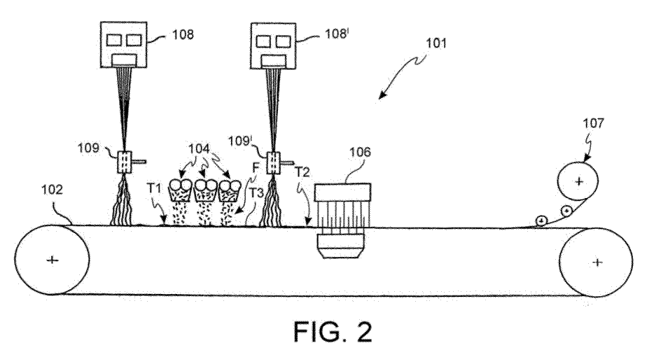

FIG. 2 shows a different embodiment, wherein a tri-layered material is obtained by producing in-line the spunbond continuous filaments.

On a transporting means 102, such as a conveyor belt, a layer T1 of spunbond continuous filaments may be laid after having been extruded through a spinneret 108 coupled to a suction fan 109, according to a conventional technique.

Subsequently, on the said layer T1 water-absorbent fibres F, such as cellulose pulp fitires, may be laid through suitable dispensing means 104 to form a layer T3.

After the deposition of said water-absorbent fibres, a second layer T2 of spunbond continuous filaments may be extruded from a second spinneret 108′-suction fan 109′ and laid over the previous layers.

The tri-layer material then may be passed through dry bonding means 106 (shown in FIG. 2 as needle-punching means) and may be finally wound on a receiving bobbin 107.

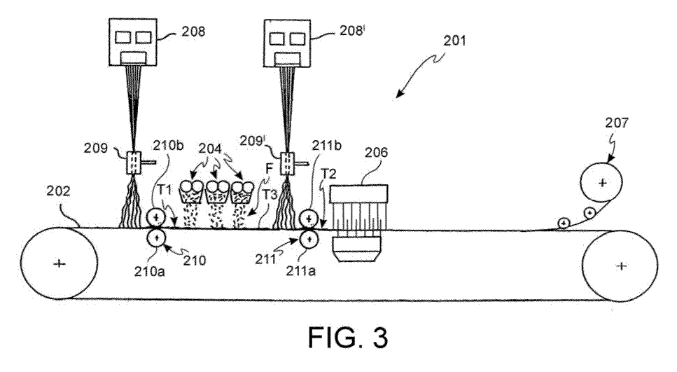

FIG. 3 shows another embodiment that differs from the embodiment of FIG. 2 in that, after the deposition of both the first layer T1 and the second layer T2 of spunbond continuous filament, consolidation or pre-consolidation takes place.

On a transporting means 202, such as a conveyor belt, a layer T1 of spunbond continuous filaments may be laid after having been extruded through a spinneret 208 coupled to a suction fan 209, according to a conventional technique.

Layer T1 of spunbond continuous filaments, supported by the transporting means 202, may be passed through first consolidation or pre-consolidation means 210, comprising two rollers 210a, 210b coupled in such a way as to allow layer T1 to be pressed therebetween. These consolidation or pre-consolidation means 210 can include, for example, a compactor, an embosser or a calender, as described above.

Subsequently, on the said layer T1 water-absorbent fibres F, such as cellulose pulp fibres, may be laid through suitable dispensing means 204 to form a layer T3.

After the deposition of said water-absorbent fibres, a second layer T2 of spunbond continuous filaments may be extruded from a second spinneret 208′-suction fan 209′ and laid over the previous layers.

The layered material, supported by the transporting means 202, may be passed through second consolidation or pre-consolidation means 211, comprising two rollers 211a, 211b coupled in such a way as to allow the layered material to be pressed therebetween. These consolidation or pre-consolidation means 211 can include, for example, a compactor, an embosser or a calender, as described above.

The tri-layer material then may be passed through dry bonding means 206 (shown in FIG. 3 as needle-punching means) and is finally wound on a receiving bobbin 207.

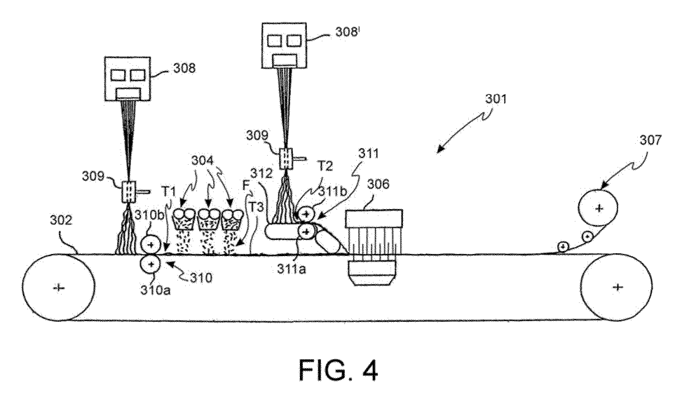

A further embodiment shown in FIG. 4 differs from the embodiment in FIG. 3 in that the second layer of spunbond continuous filaments is independently consolidated or pre-consolidated before laying it over the other layers of nonwoven material.

On a transporting means 302, such as a conveyor belt, a layer T1 of spunbond continuous filaments may be laid after having been extruded through a spinneret 308 coupled to a suction fan 309, according to a conventional technique.

Layer T1 of spunbond continuous filaments, supported by the transporting means 302, may be passed through first consolidation or pre-consolidation means 310, comprising two rollers 310a, 310b coupled in such a way as to allow layer T1 to be pressed therebetween. These consolidation or pre-consolidation means 310 may include, for example, a compactor, an embosser or a calender, as described above.

Subsequently, on the layer T1 water-absorbent fibres F, such as cellulose pulp fibres, may be laid through suitable dispensing means 304 to form a layer T3.

After the deposition of said water-absorbent fibres, a second layer T2 of spunbond continuous filaments may be extruded from a second spinneret 308′-suction fan 309′ and laid over transporting means 312 positioned above the transporting means 302 carrying the already deposited layers T1 and T3. The said transporting means 312 may be coupled to consolidation or pre-consolidation means 311, similar to the ones described above and comprising two rollers 311a, 311b of a compactor, embosser or calender. The second layer T2 of spunbond continuous filaments thus may be consolidated or pre-consolidated and then laid over the previous layers passing therebelow.

The layered material so obtained, supported by the transporting means 302, then may be passed through dry bonding means 306 (shown in FIG. 4 as needle-punching means) and finally may be wound on a receiving bobbin 307.

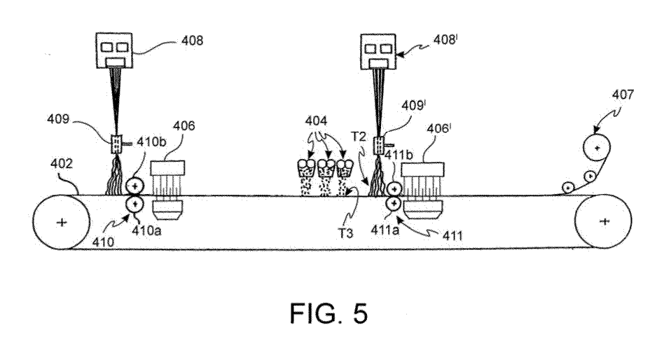

The embodiment shown in FIG. 5 differs from the embodiment of FIG. 4 in that the first layer T1 of spunbond continuous filaments is bonded before the deposition of the other layers.

On a transporting means 402, such as a conveyor belt, a layer T1 of spunbond continuous filaments may be laid after having been extruded through a spinneret 408 coupled to a suction fan 409, according to a conventional technique.

Layer T1 of spunbond continuous filaments, supported by the transporting means 402, may be passed through first consolidation or pre-consolidation means 410, comprising two rollers 410a, 410b coupled in such a way as to allow layer T1 to be pressed therebetween. These consolidation or pre-consolidation means 410 may include, for example, a compactor, an embosser or a calender, as described above.

After having been consolidated or pre-consolidated, the layer T1 may be bonded by passing it through dry bonding means 406 (shown in FIG. 5 as needle-punching means).

Subsequently, on the said layer T1 water-absorbent fibres F, such as cellulose pulp fibres, may be laid through suitable dispensing means 404 to form a layer T3.

After the deposition of said water-absorbent fibres, a second layer T2 of spunbond continuous filaments may be extruded from a second spinneret 408′-suction fan 409′ and laid over the previous layers.

The layered material, supported by the transporting means 402, may be passed through second consolidation or pre-consolidation means 411, comprising two rollers 411a, 411b coupled in such a way as to allow the said layered material to be pressed therebetween. These consolidation or pre-consolidation means 411 may include, for example, a compactor, an embosser or a calender, as described above.

The tri-layer material then may be passed through second dry bonding means 406′ (shown in FIG. 5 as needle-punching means) and finally may be wound on a receiving bob-bin 407.

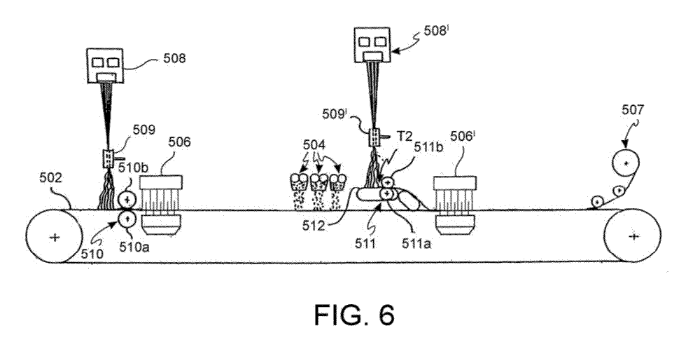

The embodiment shown in FIG. 6 differs from the embodiment of FIG. 5 in that the first layer T1 of spunbond continuous filaments is bonded before the deposition of the other layers.

On a transporting means 502, such as a conveyor belt, a layer T1 of spunbond continuous filaments may be laid after having been extruded through a spinneret 508 coupled to a suction fan 509, according to a conventional technique.

Layer T1 of spunbond continuous filaments, supported by the transporting means 502, may be passed through first consolidation or pre-consolidation means 510, comprising two rollers 510a, 510b coupled in such a way as to allow the said layer T1 to be pressed therebetween. These consolidation or pre-consolidation means 510 may include, for example, a compactor, an embosser or a calender, as described above.

After having been consolidated or pre-consolidated, the layer T1 may be bonded by passing it through dry bonding means 506 (shown in FIG. 6 as needle-punching means).

Subsequently, on the said layer T1 water-absorbent fibres F, such as cellulose pulp fibres, may be laid through suitable dispensing means 504 to form a layer T3.

After the deposition of said water-absorbent fibres, a second layer T2 of spunbond continuous filaments may be extruded from a second spinneret 508′-suction fan 509′ and laid over transporting means 512 positioned above the transporting means 502 carrying the already deposited layers T1 and T3. The said transporting means 512 may be coupled to consolidation or pre-consolidation means 511, similar to the ones described above and comprising, for example, two rollers 511a, 511b of a compactor, embosser or calender. The second layer T2 of spunbond continuous filaments is thus consolidated or pre-consolidated and then laid over the previous layers passing therebelow.

The layered material so obtained, supported by the transporting means 502 then may be passed through second dry bonding means 506′ (shown in FIG. 6 as needle-punching means) and may be finally wound on a receiving bobbin 507.

The invention also includes products for household or personal care made of the non-woven fabric material described above. Non-limiting examples of such products include cloths, rags, wipes and similar fabrics.

These products may be dry or substantially dry, therefore they should be moistered before use. Once the product has been used, it may be conveniently disposed and flushed since the materials are water-soluble or water dispersible and generally biodegradable.

Embodiments of the invention include products which are water-soluble or water-dispersible at room temperature, i.e. generally at or below about 25° C.

1. A nonwoven fabric material comprising two external layers which comprise continuous filaments of a water-soluble or water-dispersible polymer and at least one internal layer of a water-absorbent, flushable material
wherein the nonwoven fabric material comprise a water content of less than about 10% by weight and wherein it also comprises dry substances selected from the group consisting of: household or personal care items, substances for topic medical use and/or microcapsules for slow releasing of one or more of such substances,
wherein said water-absorbent material comprises cellulose pulp fibres, and wherein said nonwoven fabric material is impregnated with a hydrophobic substance which is a solution of a derivative of a fatty acid in a non-aqueous organic solvent.

2. The nonwoven fabric material of claim 1, comprising a dry tri-layered material which comprises one internal layer of cellulose pulp fibres, one upper layer of said continuous filaments of a water-soluble or water-dispersible polymer and one lower layer of said continuous filaments of a water-soluble or water-dispersible polymer.

3. The nonwoven fabric material of claim 1, wherein the said cellulose pulp fibres have lengths that vary from one another less than about 2.5 mm.

4. The nonwoven fabric material of claim 1, wherein the said water-soluble or water-dispersible polymer is selected from the group consisting of: polyvinyl alcohol (PVA), carboxymethyl cellulose, Guar Gum, alginate, starch, a water-dispersible co-polyester and mixtures thereof

5. The nonwoven fabric material of claim 1, wherein the said continuous filaments have a diameter between 0.5 dtex and 6.7 dtex.

6. The nonwoven fabric material of claim 1, wherein the said substances for house-hold items are selected from the group consisting of: dry detergents, surfactants, waxes for ceramic or wooden floors, waxes for wood furniture surfaces, surface disinfectants, anti-bacterial products, antiviral products, antifungal products for household use, metal or wood polishing creams and cleaning formulations.

7. The nonwoven fabric material of claim 6, wherein the dry detergent is sodium alfa-olefin (C14-C16) sulphonate.

8. The nonwoven fabric material of claim 1, wherein the said substance for personal care are selected from the group consisting of: formulations for personal hygiene, sanitization, skin creams, waxes, tanning creams, sunscreen formulations, insect repellent formulations, deodorants, perfumes, antibacterial formulations, antiviral formulations, antifungal formulations, make-up removing substances and cosmetic products in general.

9. The nonwoven fabric material of claim 1, wherein the substances for topic medical use are selected from skin disinfectants, skin antibacterial agents, antiviral substances, antifungal substances, cicatrizing formulations, any drug formulation that can be administered by topical application, and sanitizing compositions for medical facilities, appliances and devices.

10. The nonwoven fabric material of claim 1, comprising super-absorbent fibres and/or super-absorbent polymer powder.

11. The nonwoven fabric material of claim 10, wherein the said super-absorbent polymers are selected from the group consisting of: poly-acrylic acid sodium salt, polyacrylamide copolymer, ethylene maleic anhydride copolymer, cross-linked carboxymethyl cellulose, polyvinyl alcohols copolymers, cross-linked polyethylene oxides and starch-grafted copolymer of polyacrilonitrile.

12. The nonwoven fabric material of claim 1, comprising one or more additive selected from the group consisting of: lubricating additives, antistatic additives, hydrophilic additives, and anti-foam additives.

13. The nonwoven fabric material of claim 1, having a weight/surface ratio (basis weight) between about 30 and about 100 g/m².

14. The nonwoven fabric material of claim 1, wherein said material is water-soluble or water-dispersible at room temperature.

15. The nonwoven fabric material of claim 1, wherein said hydrophobic substance comprises from about 0.5 to about 10 w % or about 0.6 to about 5 w %.

16. The nonwoven fabric material of claim 1, wherein the derivative of a fatty acid is stearoyl chloride.

17. A process for manufacturing nonwoven fabric material comprising the following steps:
a) placing a first layer of spunbond filaments made of a water-soluble or water-dispersible polymer;
b) placing, on the layer of step a), a layer of water-absorbable fibres;
c) placing another layer of said spunbond filaments;
d) bonding the single layers and/or the multi-layered material at a dry state, to provide one or more bonding step, wherein the one or more bonding step is performed in such a way that the so-obtained nonwoven fabric material has a water content of less than about 10% by weight.
e) impregnating the nonwoven fabric material of step d) with dry substances selected from the group consisting of: household items, personal care items, dry substances for topic medical use, and microcapsules for slow releasing of one or more of such substances;
f) impregnating the nonwoven fabric material with a solution of a derivative of a fatty acid in a non-aqueous organic solvent.

18. The process of claim 17, comprising one or more steps of consolidation or pre-consolidation of the single layers of continuous filaments and/or of the multi-layered material before the one or more bonding step.

19. The process of claim 17, wherein the said spunbond filaments are produced in line with the formation of the said nonwoven fabric material or are made on a separate production line.

20. The process of claim 17, wherein the said consolidation or pre-consolidation of the single layers and/or of the multi-layered nonwoven fabric material is accomplished by passing the layered material through two rollers of a compactor, embosser or calendar.

21. The process of claim 17, wherein the said step of bonding of the single layers and/or of the multi-layered material comprises a dry-laid process.

22. The process of claim 17, wherein the said step of bonding of the single layers and/or of the multi-layered material comprises a chemical bonding process.

23. The process of claim 17, wherein the said step of bonding of the single layers and/or of the multi-layered material comprises a needle-punching process.

24. The process of claim 17, comprising one or more steps of adding to the single layers and/or to the nonwoven fabric material, before or after the said one or more bonding step, one or more substances selected from the group consisting of: household items, personal care items, topic medical formulations, super-absorbent fibres, super-absorbent polymer powder and additional substances.

25. The process of claim 17, comprising steps selected from the group consisting of:
colouring, finishing of a chemical nature as the anti-pilling treatment, hydrophilic treatment, antistatic treatment, improvement of flame proof properties, substantially mechanical treatments and any combination of the foregoing.

26. The process of claim 17, wherein the step of treating the said nonwoven fabric material with a solution of a hydrophobic substance in a non-aqueous organic solvent is performed with a toluene solution of stearoyl chloride.

27. A product for household or personal care, comprising the nonwoven fabric material of claim 1.

28. The product of claim 27, wherein said product is selected from the group consisting of: a cloth, a rag or a wipe.
